(19) United States
(12) Patent Application Publication
(10) Pub. No.: US 2023/0190125 A1
(43) Pub. Date: Jun. 22, 2023

(54) System and Method for Detecting Coughs from Sensor Data

(71) Applicant: LifeQ B.V., Amsterdam (NL)

(72) Inventors: Tom deLaubenfels, Atlanta, GA (US); Franco du Preez, Cumming, GA (US); Theo Wikus Villet, Kuilsriver (ZA); Laurence Richard Olivier, Alpharetta, GA (US)

(21) Appl. No.: 17/999,262

(22) PCT Filed: Jun. 1, 2021
(86) PCT No.: PCT/US2021/035209

§ 371 Date: Nov. 18, 2022

Related U.S. Application Data
(60) Provisional application No. 63/032,212, filed on May 29, 2020.

(51) Int. Cl. A61B 5/0295; A61B 5/08

(52) U.S. Cl. CPC A61B 5/0295; A61B 5/0823

(57) ABSTRACT

A method of detecting coughs using photoplethysmography sensor data is presented. In some embodiments, accelerometry sensor data may also be used in conjunction. A system is presented for the purpose of automatic cough detection, which may also incorporate auxiliary data relevant to the occurrence of coughs. Auxiliary data, may be used to improve cough detection and/or be for contextualization and categorization of detected coughs.

### BACKGROUND

Coughing is a protective reflex through which the human body attempts to clear the respiratory passages of irritants, foreign particles, fluids, and microbes. Coughs are typically initiated with an inhalation, followed shortly by a forceful exhalation against a closed glottis and subsequent opening of the glottis; the result is a rapid release of air from the lungs which is usually accompanied by a distinct sound. Frequent or chronic coughing can indicate the presence of a disease; the development of a frequent or chronic cough is often an indicator of disease-onset, while a worsening cough is often indicative of disease progression and/or declining health. Such diseases are often the result of infection by contagious pathogens such as, notably, SARS-CoV-2 (COVID-19). Coughing can also result from exposure to environmental particulates such as smoke, pollution, pollen, dust, etc., which can have similar impacts to health as many internal diseases.

Coughing also occurs commonly for reasons that are relatively benign, such as mild irritation of the respiratory passages due to odors, acute exposures to allergens such as dust and dander, food or phlegm in the throat, etc. For healthy individuals experiencing the onset of disease/infection, or encountering hazardous environmental particulates such as smoke, silica dust, etc., without awareness of their condition or exposure, it may be difficult to identify malign coughs from benign ones.

There are known tools that can be used for cough detection. Such devices include spirometers, electrocardiogram sensors, chest belts, oximeters, and microphones. However, these devices can be invasive and do not have proven user tolerance. In addition, microphones, which may be the most common tool for automated detection currently in use, are subject to environmental noise.

Therefore, there is a need to be able to identify malign coughs from benign ones. In addition, the devices used to measure and identify such coughs need to be non-invasive and have high user-tolerance. Further, such devices should not be subject to environmental noise.

### SUMMARY

The invention presented describes a method of detecting coughs using photoplethysmography (PPG) and accelerometry sensors worn on or placed near the body. In some embodiments, the invention presented may also consider “Auxiliary” data both physiological and non-physiological, in order to increase detection efficiency and specificity in the context of human activities, underlying diseases, environmental conditions, and other factors relevant to the occurrence of coughs. The Auxiliary data may be used to subsequently categorize detected coughs depending on the context in which they occur (e.g. post-exercise, during illness, high pollution levels in the region, etc.). A system for incorporating Auxiliary data from a variety of sources and performing appropriate categorization of detected coughs is also described in the present disclosure. In addition to improving detection, this same data can be used to contextualize and categorize detected coughs, providing valuable insights on both individual and population scales.

As will be shown below, there is value to be gained from detecting, contextualizing, and categorizing coughs in a manner which is automated, low-friction, and easily implementable at scale. The methods and system presented in this disclosure are advantageous for this purpose for several reasons. For one, the underlying sensors (PPG and accelerometry) are already present in a variety of commercially available devices such as smartwatches, fitness trackers, mobile phones, and body-monitoring patches. These devices are typically enabled with Internet of Things (IOT) technology, allowing for measurements which are easily trackable across a variety of platforms.

In an aspect, the invention is directed to a method to detect the occurrence of coughs, comprising the steps of collecting non-invasive signals corresponding to physiological data from a subject, processing the collected signals to generate physiological data associated with the subject, and detecting, from the physiological data, physical acts of coughing. The physical acts can include, but are not limited to, the physical act comprises inhalation, exhalation against closed glottis, opening of glottis, or relaxation. The non-invasive signals can be collected by photoplethysmography (PPG) sensors, which allows the monitoring of blood volume changes to detect physical acts of coughing. In some aspects, the non-invasive signals include accelerometry signals associated with accelerometry data of the subject. Identifying data indicating a physical act of coughing is done by signal magnitude thresholding, signal magnitude deviations outside of statistical norms, time-domain analysis methods, frequency-domain analysis methods, signal decompositions, statistical methods (e.g., autocorrelations), or machine learning methods (e.g., recurrent and convolutional neural networks).

In some aspects, the method can incorporate auxiliary data to provide context on conditions under which the non-invasive signals are collected, thus allowing cough detection techniques to be modified or temporarily paused. Auxiliary data refers to any information which has relevance to the occurrence and/or detection of coughs. The auxiliary data can include data relevant to signal quality as a result of varying measurement conditions. Examples include, but are not limited to: knowledge that a subject is under motion, knowledge that a subject is asleep, etc. Data of the physiological state of the subject can include knowledge that a subject has recently exercised, knowledge that a subject is sick or recovering from illness, and information on potential hazards in a subject's environment e.g. air pollution. The auxiliary data is used to change cough detection techniques to best suit measurement conditions, adjust detection parameters to be more or less sensitive, or pausing detection efforts when measurement conditions are poor or too highly confounded. The auxiliary data can also be used to contextualize and/or categorize detected coughs. Examples of the application of such information includes, but are not limited to: identifying coughs which may indicate the onset or worsening of a disease, identifying coughs which may indicate or quantify the severity of exercise-induced asthma, and identifying public impacts of environmental hazards e.g. air pollution.

In an aspect, the invention is directed at a system for automatically detecting coughs of a subject that includes sensors and a processor. These components, as well as others, can be distributed across any suitable combination of IOT-enabled devices (such as PPG-enabled smartwatches or other body-monitoring devices, mobile phones, personal computers, or cloud servers) in order to form a deployable cough detection solution which can monitor arbitrary users. The sensors are configured to collect non-invasive physiological signals associated with the subject. The sensors can include PPG sensors and accelerometers. The processor can be configured to detect occurrences of coughs automatically via changes in input sensor data. The processor is configured to process the non-invasive physiological signals into physiological data related to the subject, detect occurrences of coughs from changes in the physiological data, and generate a cough event output upon detection of a cough. In some aspects, the cough events include a flag or other indication that a cough has been detected, a timestamp which marks the point in time at which the cough was detected, and/or a confidence value. The confidence value may be qualitative (e.g. low/medium/high) or quantitative (e.g. a value in the range [0, 1]). The cough events can also include a measurement of cough intensity, which may be qualitative (e.g. low/medium/high) or quantitative (e.g. a value in the range [0, 1]). The system can be utilized to monitor multiple subjects and aggregate coughing events in order to enable population-scale studies. The system can utilize a user interface to show detected cough events, or summary metrics thereof, to the subject or authorized third parties (e.g., physicians, etc.).

In some instances, the user interface can be utilized to request a subject to input information pertaining to the user, including demographics, general health, medical history, illness symptoms. Follow up questions to clarify collected data, as well as to obtain additional information, can be asked via the user interface. The collected data can be used by an auxiliary data module configured to utilize auxiliary data to generate context of measurement conditions or coughs. In some instances, the context of the measurement conditions can lead the processor to modify the detection. The detection of cough events, and the modification of such detection, can be done by an algorithm. The algorithm can include any combination of selection rules, mathematical techniques or functions, machine learning methods, or other techniques, which makes decisions on how to modify cough detection efforts based on contextual information derived from the input data. The auxiliary data can be utilized by an auxiliary data module to make these adjustments. The auxiliary data module can also modify the confidence values when applicable. The auxiliary data module can categorize the cough events based on contextual information extracted. In some embodiments, a sorting module applies confidence value modifications and cough event categorizations. The auxiliary data, cough detection, and sorting modules can operate together or independently of one another.

These and other objects and advantages of the invention will become apparent from the following detailed description of the preferred embodiment of the invention. Both the foregoing general description and the following detailed description are exemplary and explanatory only and are intended to provide further explanation of the invention as claimed.

The accompanying drawings are included to provide a further understanding of the invention and are incorporated in and constitute part of this specification, illustrate several embodiments of the invention that together with the description serve to explain the principles of the invention.

### BRIEF DESCRIPTION OF THE DRAWINGS

FIGS. 1a-b illustrate trace of photoplethysmography and tri-axial accelerometry signals during 3 successive coughs under controlled conditions (subject lying down, motionless).

FIG. 2 is a block diagram indicating the different electronic devices that make up the system. Modules outlined with dashed lines can be implemented on any of the devices in the system.

FIG. 3 is a block diagram illustrating the 3 principal modules of the system (Cough Detection (105), Auxiliary Data (106), Sorting (107)) and the manner in which they exchange data.

### DEFINITIONS

PPG—photoplethysmography
IOT—Internet of things
SpO₂—blood oxygen saturation

### DETAILED DESCRIPTION

### Principles of Cough Detection Via PPG

The method of cough detection presented in this disclosure looks for changes in photoplethysmography (PPG) and accelerometry signals which are captured by sensors worn on or placed near the body. This combination of sensors is commonly found in smartwatches and fitness trackers, fingertip pulse oximeters, as well as in some mobile phones and novel body-monitoring devices such as wearable sensor patches. The sensor-carrying device will be referred to as the PPG-enabled device (100), but it should be understood that these different sensor types may not necessarily be bundled in the same device in some embodiments—for example, the PPG-enabled device might be a wearable band placed on the wrist, while accelerometry is captured by another device worn on or placed near the body (such as a mobile phone). Furthermore, because cough-induced changes to PPG signals are typically the more distinct than those in accelerometry signals, accelerometry may be excluded altogether in some embodiments of the presented method. The principles described herein for PPG-based cough detection may be applied to any sensor data which is capable of making measurements of pressure or volume dynamics in the human arterial system and/or associated body tissue.

PPG is a non-invasive optical technique used to detect volumetric changes in blood circulation. In brief one or more light-emitting diodes are placed at the surface of the skin, in conjunction with one or more photodetectors (photodiodes, CMOS, or other light-detecting sensors) which absorb light reflected by blood and tissue. In some configurations, the light-emitting diodes and photodetectors may be placed with a portion of the body between them, such as a finger. The measurement principles are the same regardless of configuration: oxygenated blood will absorb the emitted light at a different rate than skin, muscle tissue, bone, etc. It is known to those skilled in the art that, as a consequence, certain fluctuations in the quantity of light which reaches the photodetector sensor(s) (having either been reflected by blood/tissue or transmitted through it, depending on the measurement configuration) will correspond to the changing volume of blood in circulation “within range of” the sensor(s). This technique is commonly employed in a wide variety of commercial and medical devices to enable heart rate sensing, blood oxygen saturation, and other measurements. Said devices often include accelerometer sensors in addition to PPG. Besides providing an independent measurement of a subject's motion/activity levels, which is particularly valuable in the context of continuous physiological monitoring (e.g., with smart watches, fitness trackers, and other non-invasive devices), accelerometry measurements can be used to correct for motion-induced artifacts in PPG signals.

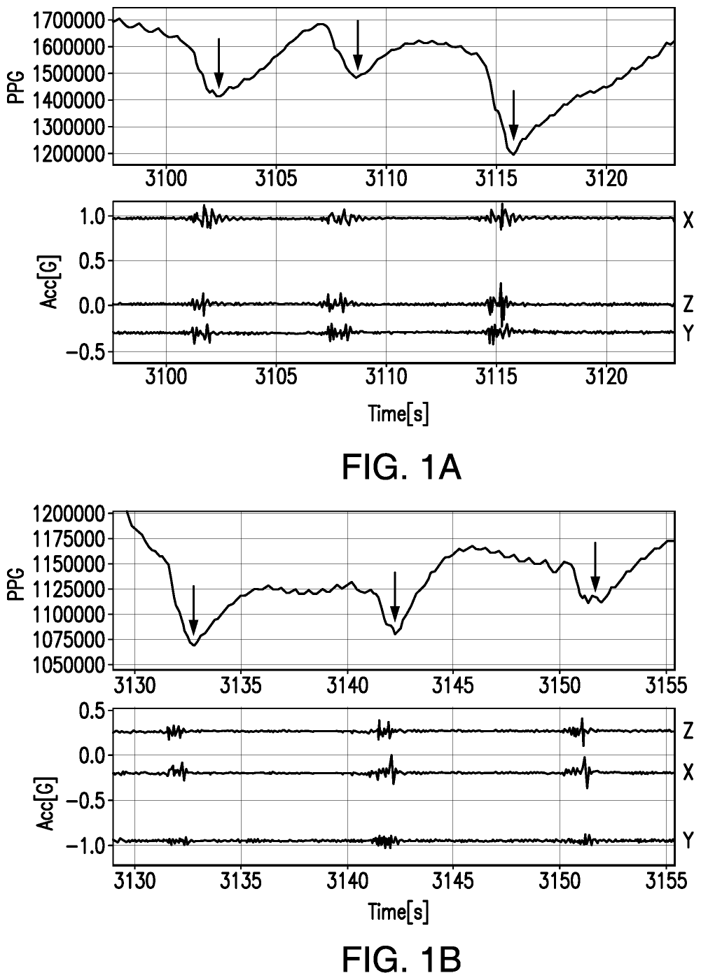

FIGS. 1a-b show two example traces of PPG and tri-axial accelerometry signals captured by a wrist-worn device under controlled conditions (i.e., subject lying down and motionless, as during sleep). The x-axis tracks time. In both FIGS. 1a-b, the y-axis is proportional to the photodiode current measured and pulsates approximately once every second due to the heartbeat of the subject, decreasing sharply with the onset of each pulse as blood flows to the skin and absorbs more light. The y-axis of the bottom-most plot (“Acc [G]”) measures the magnitude of acceleration measured by the tri-axial sensors. The data in FIG. 1 spans a period of ˜24 seconds. In both examples, 3 individual coughs occur in short succession, which can be seen clearly as perturbations in the accelerometry signals during the otherwise motionless conditions. These perturbations will primarily correspond to the forceful exhalation and subsequent opening of the glottis, leading to the characteristic action/sound of a typical cough. In conjunction with these perturbations, a temporary but significant decrease in the PPG signal can be seen (indicated with arrows), due to the underlying physiology of a coughing cycle. Forceful exhalation against the closed glottis can also lead to an acute increase in circulatory blood pressure, which can also modulate the PPG signal. Irrespective of the physiological mechanism behind the observed decrease in PPG, which might be attributable to changes in the volume of blood in the light path of the photodiode, it is a clear measurable signal of potential utility in cough detection. In addition, the magnitudes of both the PPG and accelerometry signal fluctuations in response to a cough may potentially be used as a practical measure of the cough's intensity or forcefulness, namely as an index which may be presumed to represent the intensity of muscle contraction in the chest cage which produces the cough's forceful exhalation, in addition to the force of the exhalation against the closed glottis. While this manifestation of cough intensity/forcefulness in the measured signals may differ from subject to subject depending on their physiology, it may be a useful measure to categorize the severity of coughs on an intra-subject level in order to track the progression of the disease or underlying condition leading to coughing.

The magnitude and duration of PPG fluctuations seen in FIGS. 1a-b makes them distinct from the normal sinus arrhythmia, which are regular and normal modulations in the PPG signal for a healthy individual. As such, these fluctuations may be used to determine the occurrence of coughs. Accompanying perturbations in the accelerometry signals may also be used to confirm the presence of a detected cough, and/or to increase the detection confidence in a quantifiable or qualitative manner. The cough-induced fluctuations in the PPG and accelerometry signals may be directly and automatically detected using a number of techniques, such as but not limited to: signal magnitude thresholding, signal magnitude deviations outside of statistical norms, time-domain analysis methods, frequency-domain analysis methods, signal decompositions, statistical methods e.g. autocorrelations, and machine learning methods e.g. recurrent and convolutional neural networks. Where applicable, these techniques may be applied in real time or near-real time to streaming PPG and/or accelerometry data in order to provide continuous detection of coughs. These techniques may also be applied to historical PPG and/or accelerometry data which is aggregated over an arbitrary duration of time, in order to retroactively detect coughs within some prior period. The same techniques may be applied to various secondary transformations of the PPG signal, such as but not limited to derivatives, integrations, filterings, entropic measurements, combinations, or the application of arbitrary functions, which may contain additional measurable information pertaining to the presence of coughs in the PPG signal. The invention considers any measurable change in PPG and/or accelerometry signals, or their transformations, which can be attributed to the physiological action of a cough.

In the examples of FIGS. 1a-b, the PPG signal is derived from a single light-emitting diode with a wavelength of approximately 525 nm (green light). It should be understood that the same principles of cough detection can be applied to any configuration of PPG techniques, such as those incorporating red or near-infrared light (common in commercial oximeters, for instance) and/or those which combine multiple wavelengths of light in conjunction in order to measure different reflection coefficients in the human tissue. Depending on the configuration of light-emitting diodes and photosensors, the exact nature of the changes seen in PPG signals resulting from coughs may not explicitly follow those in FIGS. 1a-b (for instance, there may be a signal increase rather than decrease in a configuration in which increased blood volume leads to diminished light absorption). Similar considerations apply to cough-induced perturbations in accelerometry signals. For instance: in the examples of FIGS. 1a-b, there are 3 axes of accelerometry (labeled X, Y, Z), but the same principles apply for configurations in which there may be only (say) a single channel of accelerometry measuring absolute magnitude. As another example, the accompanying accelerometry may be measured by sensors in a mobile phone rather than on a wrist-worn device, in which case the perturbations may appear different. In general, it should be understood that the method presented in this disclosure considers all such possibilities.

### Supporting System for Cough Detection

This disclosure also presents a system which assists the automatic detection of coughs in the method described, as well as to contextualize and categorize them, in accordance with “Auxiliary” data derived from various sources. In the context of this disclosure, Auxiliary data broadly refers to any data which is relevant to the detection and/or occurrence of coughs in any particular individual (hereafter referred to as the “user”). Auxiliary data can be collected from sensors, or other data collecting services or devices, as discussed below.

As an example: consider a scenario in which a user is undergoing physical motion, such as that which occurs during walking or other forms of exercise; this knowledge constitutes Auxiliary data and may be obtained via any applicable method, e.g. by monitoring for associated changes in one or more physiological sensors. Pertaining to cough detection, the physical motion might lead to additional variations in either or both the PPG and accelerometry signals which may resemble or confound the cough-induced fluctuations previously described. In such a scenario, the Auxiliary data can act in a de-confounding role by triggering the cessation of automatic cough detection methods for the duration of the confounding motion; this would likely lead to increased specificity of the cough-detection algorithm overall by turning off the algorithm during the walking phase. The same Auxiliary data can act in a supporting role for cough-detection in this scenario: upon the cessation of motion, the system may determine that the user has undergone a period of vigorous activity, and subsequently resume automatic cough detection with an increased sensitivity due to the likelihood of exercise-induced bronchoconstriction.

Furthermore, the coughs following exercise in this scenario would have an underlying cause which is distinct and relatively benign with respect to causes such as illness, environmental irritants, etc. In light of this, the Auxiliary data may be used to categorize detected coughs accordingly, such that the user can benefit from this knowledge. For example, simple algorithms may be employed which count the frequency of detected coughs occurring during (if the algorithm is not turned off during exercise), or shortly after, an exercise session; a relatively high frequency of exercise-induced coughs might be reported to the user (via, for example, a user interface such as the one will be described forthwith).

This scenario should be considered exemplary and by no means comprehensive. Other examples of Auxiliary data derived from physiological sensors include, but are not limited to, motion detections, activity/exercise detections, sleep detections, illness/infection presence (e.g., COVID-19, bronchitis, etc.), oxygen saturation, and breathing rate; examples of Auxiliary data derived otherwise include, but are not limited to, user annotations of illness, user disclosures of medical conditions and/or allergies, epidemiology data (e.g. outbreaks of illness in the vicinity of a user), GPS data (e.g. indications that a user has traveled), weather data, air pollution data, and regional pollen data. This disclosure considers any and all applicable Auxiliary data which may be relevant to the detection, contextualization, and categorization of coughs.

In some embodiments of the proposed system, detected coughs and accompanying Auxiliary data may be aggregated and analyzed on a population-scale. For example, an increased prevalence of detected coughs within a geographical region may indicate the outbreak of a contagious infection. In another example, the increased prevalence may accompany environmental conditions such as high air pollution, wildfire smoke, etc., which are having a detrimental effect on the health of the affected population. The invention claimed in this disclosure includes the use of Auxiliary data in conjunction with cough detection in the manner described in order to provide population-scale insights which may be actionable, e.g. by individuals, public health officials, epidemiologists, government officials, etc.

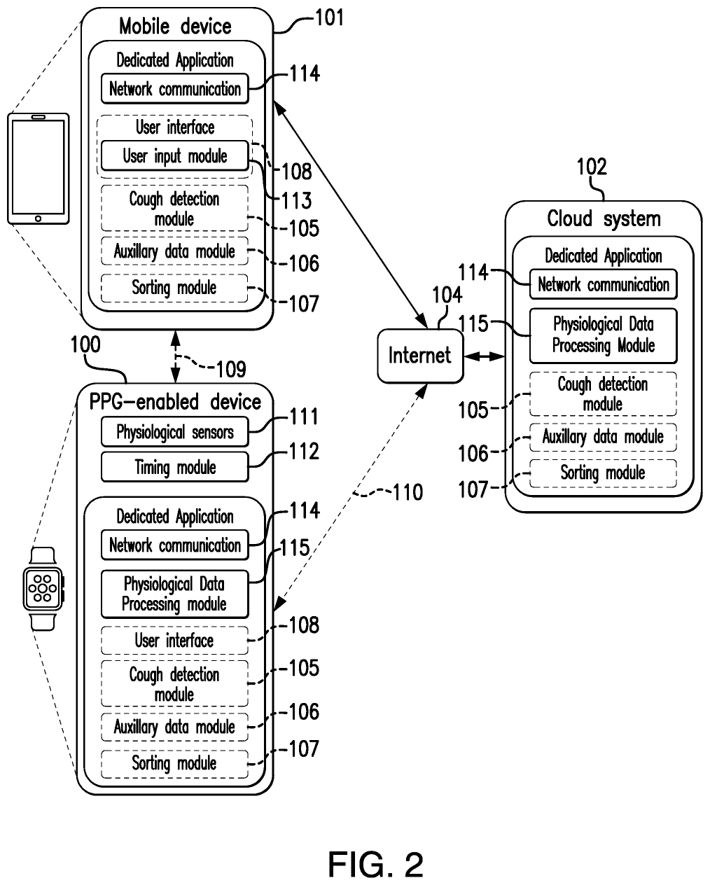

FIG. 2 is a block diagram indicating the different electronic devices 100, 101, 102 that constitute the disclosed system, as well as the modules responsible for the execution of the disclosed method and system. The principal modules related to the disclosed invention are the Cough Detection Module (105), Auxiliary Data Module (106), Sorting Module (107), and Physiological Data Processing Module (115) which is described in detail below. Also of note is a User Interface module (108) in which the user may receive information about their detected coughs, and through which they may, in certain embodiments, enter annotation data (e.g. current presence of illness) or other relevant information which may constitute Auxiliary data within the system. In the embodiment considered in FIG. 2, a PPG-enabled device (100) acts as the sensor platform, and works in conjunction with a Mobile Device (101) and Cloud System (102). The Cloud System (102) is any computer, server, or collection thereof, which exists in support of multiple users, each with one or more PPG-enabled devices (100) and/or Mobile devices (101) which communicate with the Cloud System (102) via a network connection (e.g., an internet connection) (104) in conjunction with on-board Network Communication modules (114). In the case of the PPG-enabled device 101, the Internet connection (104) may be made directly (110), e.g. by LTE connectivity or other means, or indirectly via use of the Mobile device (101) as proxy. Communication between the PPG-enabled device (100) and Mobile device (101) occurs typically through direct short-range communication (109), e.g. via Bluetooth connectivity, NFC, a connection through a local area network, or other various known communication means.

The principal system modules (105), (106), (107), and (115) may be distributed across the PPG-enabled device (100), Mobile device (101), and Cloud System (102) in any combination. For example: if the PPG-enabled device (100) is a modem smartwatch with ample computing power and a suitable interface, the system modules may well be run directly on the device. In such an embodiment, the Mobile device (101) may be excluded entirely. If, on the other hand, the PPG-enabled device (100) is a low-cost fitness tracker with limited resources and no interface, it may act solely as a sensor platform. In such an embodiment, all remaining functions of the system may be delegated to the Mobile device (101) and Cloud System (102) in the manner most appropriate. It should be noted that the Mobile device (101) and Cloud System (102) are not mandatory in all possible embodiments of the disclosed system; in some embodiments, a suitable PPG-enabled device (100) may act as the sole device in the system when it includes sufficient computing and storage capacity to host all of the principal modules (105), (106), (107), and (115)—however, in such an embodiment certain elements of the disclosed invention may not be possible, e.g. the inclusion of external Auxiliary data (weather, pollution, regional epidemiology, etc.) or the means to aggregate and analyze user cough and Auxiliary data on a population-level scale.

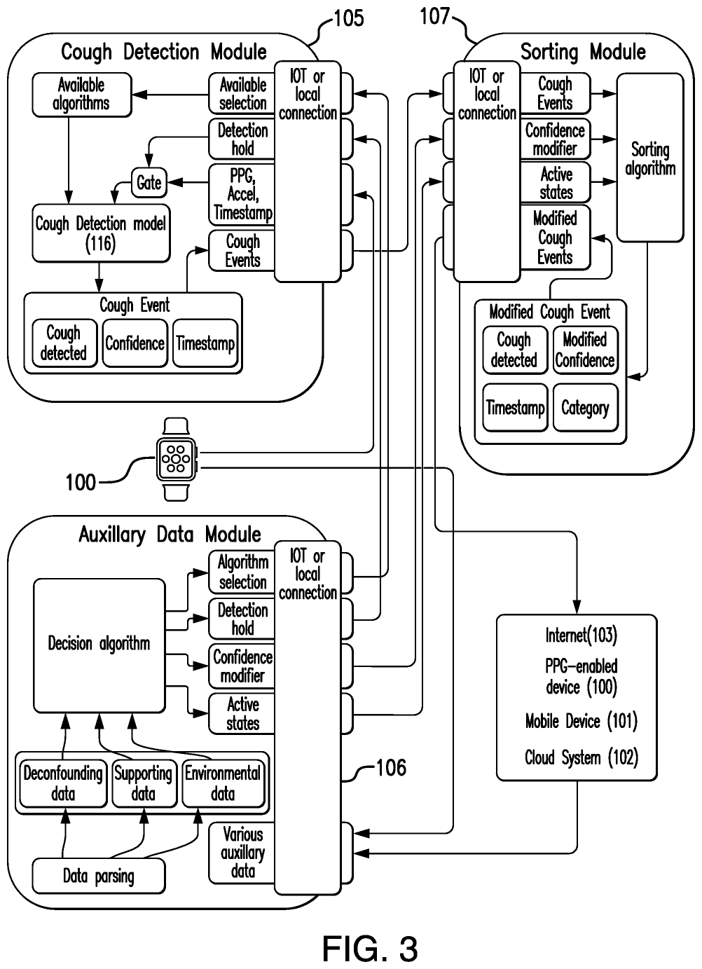

FIG. 3 is a block diagram which illustrates in greater detail the Cough Detection Module (105), Auxiliary Data Module (106), and Sorting Module (107), including the manner in which they interconnect and exchange data. Ideal embodiments of the disclosed system will include all 3 modules (105). (106), and (107) working in conjunction; however, in some embodiments the Cough Detection Module (105) may operate independently. A detailed description of the illustration follows:

The Cough Detection Module (CDM) (105) receives processed and timestamped PPG and accelerometry sensor data from the PPG-enabled device (100). This data is initially acquired by Physiological sensors (111) (e.g. the PPG photodiodes and photodetectors, as well as the accompanying accelerometers) and is provided a timestamp by an accompanying Timing module (112) which is capable of temporal context. Raw data from 111 and 112 is processed into usable forms (e.g. normalization, outlier removal, secondary transformations such as derivatives, etc) by a Physiological Data Processing Module (115) which subsequently provides data to the CDM (105). In an aspect, the CDM (105) includes a cough detection model (116). The cough detection model 116 can include one or more techniques or algorithms to automatically detect cough signatures within the received sensor data. These techniques may include but not limited to: signal magnitude thresholding, signal magnitude deviations outside of statistical norms, time-domain analysis methods, frequency-domain analysis methods, signal decompositions, statistical methods e.g. autocorrelations, and machine learning methods e.g. recurrent and convolutional neural networks. Where applicable, these techniques may be applied in real time or near-real time to streaming PPG and/or accelerometry data in order to provide continuous detection of coughs; these techniques may also be applied to historical PPG and/or accelerometry data which is aggregated over an arbitrary duration of time and stored in the PPG-enabled device 100, Mobile Device 101, Cloud System 102, or any combination thereof, in order to retroactively detect coughs within some prior period. Under nominal conditions, the detection model will receive sensor data (in real time, near-real time, or in retrospect) and subsequently output detected Cough Events, which consist of a Timestamp, a “Cough Detected” flag, and in some embodiments a Confidence value. The Confidence value may be qualitative and discrete in some embodiments, e.g. low-medium/high, or quantitative and continuous in other embodiments, e.g. on a [0, 1] range. In some embodiments, the Confidence value may represent a statistical likelihood that the detected cough matches known instances of coughing in various control data; in other embodiments it may represent the statistical weight of the true-positive cough class in a detection model. In other embodiments, the Confidence value may be excluded entirely. Cough Events may also include a Cough Intensity measurement in some embodiments (not explicitly depicted in FIG. 3) by measuring the magnitude/intensity of the cough-associated response in the PPG and/or accelerometry signals. The Cough Intensity value may be qualitative and discrete in some embodiments, e.g. low/medium/high, or quantitative and continuous in other embodiments, e.g. on a [0, 1] range. The Cough Intensity value may be manifested, for example, by comparing against control data in which subjects are asked to cough with varying degrees of force in order to provide a reference; as another example, it may be manifested by accruing detected coughs across a wide number of individuals and establishing a scale based on the associated magnitudes/intensities of the accrued coughs. Detected Cough Events are the subsequent output of the Cough Detection Module (105).

In embodiments of the system which include the Auxiliary Data Module (106), the Cough Detection Module (105) may receive a “Detection hold” command from the Auxiliary Data Module (106) in circumstances where cough detection efforts should be temporarily suspended, e.g. during detected periods of intense exercise. The mechanism of the hold may be to restrict sensor data from reaching the detection model via a simple Gate function, as illustrated in FIG. 3, or alternatively to simply switch off the detection model—the former case has been illustrated as it is assumed that a well-implemented detection model will naturally suspend itself in the event of sensor data interruptions. In addition to “Detection hold” commands, the Cough Detection Module (105) may receive “Algorithm selection” instructions from the Auxiliary Data Module (106), which modifies the techniques and/or parameters used by the detection model in order to best suit the present circumstances. For example, if the Auxiliary Data Module (106) receives information that a user is asleep, it may signal the Cough Detection Module (105) to use a low-compute technique/algorithm which performs well under sleep conditions, but not otherwise; alternatively, during wake conditions with periodic and/or frequent motion, the Auxiliary Data Module (106) may signal the use of a more sophisticated detection algorithm which is better suited to handle signal noise. Algorithm parameters may also be modified accordingly, e.g. sensitivities raised or lowered depending on circumstances. Different algorithm parameters may optimize cough detection (e.g. sensitivity vs. specificity) under the various types of conditions.

The Auxiliary Data Module (106) receives as its input a variety of uncategorized Auxiliary data from either the PPG-enabled device (100), Mobile Device (101), Cloud System (102), Internet (103), or any combination thereof. For example, the input data might be processed physiological data derived from various sensors (including PPG and accelerometry), such as motion presence detection, physical activity detection, sleep detection, illness detection, etc., which was computed on any of the aforementioned platforms illustrated in FIG. 2. As another example, the input data might be entered by the user into the User Interface (108), such as annotations of illness. As yet another example, the input data might be information retrieved from the Internet (103), such as regional air pollution levels, regional pollen counts, information pertaining to the outbreak of infectious diseases, etc. These examples should not be taken as comprehensive—this disclosure considers any data relevant to the detection and/or occurrence of coughs, collected from any source, as potential Auxiliary data for the purposes of the disclosed system. The uncategorized auxiliary data will then be parsed according to the methods discussed below.

Data input to the Auxiliary Data Module (106) is parsed into one of three principal categories:
1. De-confounding data, which is used primarily to help increase detection efficiency and specificity in the cough detection model via selection of the appropriate technique/algorithm, and/or by pausing detection efforts at appropriate times. Examples include, but are not limited to, sleep or wake state classifications, detections of motion and/or activity, heart rate, and breathing rate.
2. Supporting data, which is used primarily for the contextualization of detected Cough Events. Examples include, but are not limited to, knowledge or detections of illness in the user, knowledge of pre-existing conditions in a user, knowledge or detections of post-exercise states, and various physiological data such as measured SpO₂ levels.
3. Environmental data, which is also used primarily for the contextualization of detected Cough Events. Examples include, but are not limited to, weather data, pollution levels, pollen levels, presence of smoke or other environmental hazards, and infectious disease information.

Parsing is performed primarily by identifying known data types (e.g., sleep/wake data) and/or their sources (e.g., from a sleep/wake detection algorithm). Note that the parsing is not definitive—the objective is merely to 1) streamline the subsequent steps which convert Auxiliary data into actionable modifications to the cough detection model (as explained above), and 2) more easily contextualize and categorize detected Cough Events for the sake of individual user insights and/or aggregated analytics. Once the Auxiliary data has been parsed and broadly categorized, it is passed into a Decision algorithm as shown in FIG. 3. The Decision algorithm is any combination of selection rules, mathematical techniques or functions, machine learning methods, or other techniques not explicitly named, which provide the outputs of the Auxiliary Data Module (106) as illustrated in FIG. 3. The simplest example of an appropriate Decision algorithm would be one or more decision tree models in which predefined states of interest are combined to give appropriate combinations of outputs. The outputs of the Auxiliary Data Module (106) are:
1. Algorithm selection instructions sent to the Cough Detection Module (105).
2. Detection hold commands sent to the Cough Detection Module (105).
3. Confidence modifiers for detected Cough Events, sent to the Sorting Module (107).
4. Active states to be appended to detected Cough Events, sent to the Sorting Module (107).

The Sorting Module (107) receives as inputs Cough Events from the Cough Detection Module (105), as well as the Confidence modifiers and Active state categorizations from the Auxiliary Data Module (106). Confidence modifiers are any set of instructions, mathematical functions, scalars, etc., which change the Confidence values of detected Cough Events based on context provided by relevant Auxiliary data. For example, the Auxiliary data might include knowledge that the user is currently ill, in which case the Confidence of detected Cough Events might subsequently be increased by some factor. As another example, the Auxiliary data might include an algorithm determination that the user has recently conducted intense exercise, as well as prior or learned knowledge (within the context of the disclosed system) that the same user has a propensity for coughing due to exercise-induced bronchoconstriction—this information might also subsequently increase Confidence values by a factor. Active states are, simply, the set of relevant conditions or categories under which detected Cough Events may be contextualized. Examples include, but are not limited to: physiological states such as sleep, fever, or low SpO₂; behavioral states such as post-exercise or detected stress; acute health states such as ongoing illness; environmental states in the user's region such as contagious disease outbreaks, above-normal air pollution, above-normal pollen counts, presence of smoke or other particulates, or weather context; etc.

Outputs of the Sorting Module (107) are Cough Events with a modified Confidence and added categorization, when applicable. These are returned to, and stored on, any of the system devices illustrated in FIG. 2. In embodiments in which the Sorting Module (107) and/or Auxiliary Data Module (106) are not included, the unmodified Cough Events output from the Cough Detection Module (105) are returned and stored instead.

Cough Events, or any summaries or summary metrics thereof, can be displayed to individual users via the User Interface (108). Though not explicitly illustrated in FIG. 2, the User Interface (108) may in some embodiments operate on a user's personal computer, or alternatively be accessed remotely (e.g., the User Interface is hosted on an external computer server and displayed via web-based interface). Cough Events for a particular user may, with the user's consent, be displayed to a third party such as a monitoring physician, in which case the disclosed method and system can act as a tool for health monitoring in a clinical context. Anonymized Cough Events from multiple users within the disclosed system may also be aggregated in the Cloud System (102) or other computer server for the purposes of population-scale study by third parties. As an example, an increased prevalence of Cough Events among users within a particular geographical region may indicate the spread of a contagious disease, if a more suitable explanation is not to be found within the Auxiliary data (e.g. regionally elevated air pollution levels). As another example, in regions prone to wildfires and their associated smoke, the prevalence of Cough Events among local users may be monitored by public health officials to gauge the effects of the smoke on the population. As yet another example, Cough Events may be combined with other anonymized health assessments made with physiological data captured on users, and/or with health or medical history information volunteered by users via the User Interface (108) or other means, in order to perform These examples should not be considered comprehensive—in general, this disclosure considers any aggregation and subsequent analysis of anonymized Cough Events from multiple users as being a feature of the proposed system.

Having thus described exemplary embodiments of the present invention, it should be noted by those skilled in the art that the disclosures are exemplary only and that various other alternatives, adaptations, and modifications may be made within the scope of the present invention. Accordingly, the present invention is not limited to the specific embodiments as illustrated herein, but is only limited by the following claims.

What is claimed:

1. A method to detect the occurrence of coughs, comprising:
a. collecting non-invasive signals corresponding to physiological data from a subject;
b. processing the collected signals to generate physiological data associated with the subject; and
c. detecting, from the physiological data, physical acts of coughing.

2. The method of claim 1, wherein the non-invasive signals are collected by photoplethysmography (PPG) sensors, and wherein the detecting physical act of coughing is done by monitoring blood volume changes captured by the PPG sensors.

3. The method of claim 2, wherein the non-invasive signals further comprises accelerometry signals and the processing further comprises generating accelerometry data.

4. The method of claim 2, wherein the physical act comprises inhalation, exhalation against closed glottis, opening of glottis, or relaxation.

5. The method of claim 4, wherein the identifying data indicating a physical act of coughing is done by signal magnitude thresholding, signal magnitude deviations outside of statistical norms, time-domain analysis methods, frequency-domain analysis methods, signal decompositions, statistical methods, or machine learning methods.

6. The method of claim 2, further comprising, prior to detecting and after processing, incorporating auxiliary data to provide context on conditions under which the non-invasive signals are collected, thus allowing cough detection techniques to be modified or temporarily paused.

7. The method of claim 6, wherein the auxiliary data comprises data relevant to signal quality as a result of varying measurement conditions or a physiological state of the subject.

8. The method of claim 7, wherein the auxiliary data is used to change cough detection techniques to best suit measurement conditions, adjust detection parameters to be more or less sensitive, or pausing detection efforts when measurement conditions are poor or too highly confounded.

9. The method of claim 7, wherein the auxiliary data can be used to contextualize and/or categorize detected coughs.

10. A system for automatically detecting coughs of a subject, comprising:
a. sensors configured to collect non-invasive physiological signals associated with the subject; and
b. a processor configured to:
i. process the non-invasive physiological signals into physiological data related to the subject;
ii. detect occurrences of coughs from changes in the physiological data; and
iii. generate a cough event output upon detection of a cough.

11. The system of claim 10, in which the cough events comprise:
a. a flag or other indication that a cough has been detected;
b. a timestamp which marks the point in time at which the cough was detected; and
c. a confidence value.

12. The system of claim 10, wherein the cough events further comprises a measurement of cough intensity.

13. The system of claim 10, wherein the sensors comprise a PPG sensor and an accelerometer.

14. A system of claim 10, further comprising an auxiliary data module configured to utilize auxiliary data to generate context of measurement conditions or coughs.

15. The system of claim 14, wherein the processor is configured to modify the detection based on the context of the measurement conditions.
